(19) United States
(12) Patent Application Publication
(10) Pub. No.: US 2022/0401174 A1
(43) Pub. Date: Dec. 22, 2022

(54) Skin-Tube Clamping Device

(71) Applicant: Axel Werner Fritz Blau, Genoa (IT)

(72) Inventor: Axel Werner Fritz Blau, Genoa (IT)

(21) Appl. No.: 17/777,032

(22) PCT Filed: Nov. 25, 2019
(86) PCT No.: PCT/IB2019/060112

§ 371 Date: May 16, 2022

(51) Int. Cl. A61B 90/00; A61B 17/326

(52) U.S. Cl. CPC A61B 90/02; A61B 17/326

(57) ABSTRACT

An apparatus is provided for clamping portions of the human penile shaft skin or the foreskin (30) to allow for penile skin extension through the application of an external axial force. A soft hollow body (100), at its proximal end, has a mushroom-shaped outer surface (200) and a funnel-shaped inner portion (210) that extends into a structured axial cavity. Midway, the hollow body evolves into a tube (300) towards its distal end. The structured axial cavity inside the hollow body carries a tightly inserted, rigid, hollow pulling rod (400). Upon placing the funnel-shaped portion (210) of the hollow body (100) onto the glans (20), penile skin (30) is slid onto the stipe of the mushroom-shaped outer surface until the top portion of the skin tube comes to rest in a toroidal recess (240). The skin is entrapped and held in place by folding the distal tube (300) of the hollow body (100) over. In operation, a pulling force acting on the hollow pulling rod insert (400) lifts the hollow body (100) with the entrapped skin (30) from the glans (20) and thereby stretches the penis skin.

### TECHNICAL FIELD

This presented skin-tube clamping and tensioning device relates generally to clamping devices. In particular, it relates to devices, which clamp the skin. It also relates generally to tissue traction devices, and more particularly to devices, which stretch the skin. It also relates to devices, which promote skin expansion through new skin growth.

### BACKGROUND ART

Throughout the history of mankind, there are numerous examples of persons suffering from or—conversely—desiring the enlargement of their skin. Undesirable and partially irreversible skin expansion occurs naturally through tissue tension during pregnancy and obesity.

Desirable induced skin or soft tissue expansion is practiced by various tribes, indigenous peoples and individuals of modern society to enlarge lips, nostrils or ear lobes. The natural capacity and response of the body to relieve tensile stress by growing additional soft tissue is also exploited in modern medicine for various diagnostic indications. An example for medically induced skin expansion is the surgical balloon insertion beneath the skin to more easily accommodate an implant in the formed cavity after balloon removal several days to weeks later. The concept of tissue expansion is also applied to bone lengthening in distraction osteogenesis.

Two particular examples of skin extension are the elongation of the foreskin or its reconstruction after circumcision. Major motives are the enhancement or restoration of physiological appearance, psychological and sexual well-being, self-confidence and self-esteem.

Circumcision is prevalent in many societies and regions on earth. It is still performed for religious, cultural, traditional or medical reasons. Today, only few options exist to partially reverse circumcision. While there are currently no techniques to restore or regrow a foreskin with its fully functional properties and appearance, various strategies are available to generate a surrogate foreskin.

In surgical reconstruction, a skin graft is taken from the host and inserted at a strategic place of the remaining penis skin. This is a costly, invasive and in most cases a multistep procedure. Although being the fastest with several weeks to months healing time, it may lead to post-surgery complications, requires sexual abstinence during healing, leaves a visible scar behind and is often cosmetically unsatisfactory.

### DISCLOSURE OF INVENTION

The presented skin-tube clamping and tensioning device can be applied to the penis and be pulled. It is a non-surgical apparatus for gently stretching and thereby symmetrically elongating the skin of a penis over time when an external pulling force is axially applied in a gradual manner.

### Technical Problem

To date, the majority of published, marketed or self-made non-surgical foreskin restoration devices or techniques are based on the temporary mechanical stretching or tensioning of portions of the remaining penis skin to mechanically trigger induced cell division. If properly performed by employing low to medium forces, this leads to gradual tissue expansion.

All of these devices clamp the skin in one or the other way to a pulling aid, either by mechanical entrapment, friction, adhesives or a combination thereof. Penile skin clamping devices based on these principles are also known in the field of male catheters, but are optimized for leak-free urinary drainage rather than the carriage of high tensile loads.

Means to direct and maintain the stretching force or tension are manual pulling, adhesive tape, elastic bands attached to other body parts, springs or elastic bands on or within a device, gravity through weights or inflatable balloons enveloped by a tube of remaining or already elongated skin. Over time, a sheath-like skin tube will form, partially or fully covering the glans.

Despite the variety of existing devices and techniques, they fall short in the one or the other way.

Some devices require a decent amount of time to apply or to take off.

Some of these will not allow the user to urinate without requiring them to take the device off due to the lack of a urinary passage.

Others create discomfort over prolonged wearing periods, which may result from their insufficient biocompatibility and tendency to cause skin irritation. Such discomfort may also be caused by caps or spacers that, in some devices, are pushing against the glans to counteract the exerted strain on the pulled skin tube. The close contact between such objects and the tip of the glans may furthermore carry the risk of desensitizing the glans over time or creating an undesirable bacterial microenvironment, which may cause inflammation of the urinary tract.

Discomfort may furthermore be due to the choice of too large stretching or clamping forces. Discomfort is commonly augmented by a stiffness mismatch between the tissue and the employed materials in contact with the skin. These may lead to a too tight squeezing of the entrapped tissue, thereby impeding blood circulation and metabolic action, which is signaled by the nervous system through pain. The latter is often described by users as a pinching or burning sensation. If ignored, it may result in ischemia and subsequent tissue damage.

The skin clamping mechanism of a variety of devices is opaque, which makes it furthermore difficult to judge on the proper seating and appearance of the skin underneath and to detect harmful, clamping-related problems early on.

Some devices are difficult to conceal due to their bulky or obtrusive means of internally generating or externally applying the required stretching forces. Such devices are partially revealed if not sufficiently hidden under appropriate garment and tend to scratch against other body parts in certain body postures.

Due to the choice of materials, some devices do not tolerate exposure to humidity such as body sweat, when taking a shower or during aquatic sport activities. Adhesive tape may detach, various polymer or metallic materials may slip off.

### Technical Solution

The presented skin-tube clamping and tensioning device overcomes these shortcomings and provides further related advantages, which will become apparent from the following, more detailed description.

The presented skin-tube clamping and tensioning device reliably entraps a skin tube, be it existing foreskin or skin pushed forward from the penile shaft, and holds it in place under axial tension.

In its preferred embodiment, a hollow body has a semi-rigid, mushroom-shaped outer surface at one end and a funnel-shaped inner portion that extends into a structured axial cavity. Midway, the hollow body evolves into a soft, translucent cylindrical or bell-shaped tube towards its opposite end. The structured axial cavity inside the hollow body carries a hollow and tightly anchored rigid pulling rod insert with an appendix at its end to attach a pulling aid for applying a pulling force. The hollow pulling rod insert has an inner tubular urination passage and an outer structured locking surface that matches the shape of the structured axial cavity inside the hollow body, thereby firmly locking the hollow pulling rod insert mechanically within the hollow body.

Upon placing the funnel-shaped portion of the hollow body onto the tip of the glans, remaining foreskin or skin from the penis shaft is slid onto the stipe of the mushroom-shaped outer surface until the top portion of the skin tube comes to rest in a toroidal recess. Similar to one type of male external catheter, the skin tube is entrapped and held in place by folding the soft distal tube of the hollow body over onto the skin tube. In this folded configuration, without exerting any tension onto the device, the funnel-shaped entry of the hollow body rests on the glans and thereby forms a liquid-tight seal. Upon urination, the liquid is carried through the funnel-shaped inner portion of the hollow body into the tubular passage of the rigid hollow pulling rod insert residing in the structured axial cavity of the hollow body and discharged from the hollow pulling rod insert at its opposite end.

In operation, a string or any other handle, attached to the terminal appendix of the rigid hollow pulling rod insert, is pulled, thereby lifting the hollow body with the entrapped skin from the glans and exerting a stretching force onto the penis skin. Depending on the user's personal preference, this stretching force can be maintained manually, by attaching weights to the hollow pulling rod insert or by elastic straps clamped to the hollow pulling rod insert at one end and attached to or wrapped around other body parts at their other ends.

### Advantageous Effects

With respect to foreskin extension and restoration, the presented skin-tube clamping and tensioning device introduces a one-piece, tissue friendly and robust non-surgical device for penile skin elongation that is easy to clean and to disinfect, easy and fast to both apply and take off, comfortable and unobtrusive to wear, yet very effective in generating a surrogate foreskin of natural appearance over the period of a few months. It offers the desirable features of non-surgical prior art foreskin stretching devices, but diminishes or completely abolishes their design- and construction-related shortcomings.

In particular, due to its soft, foldable sheath, the presented device can be easily applied to and remains equally well seated on both a flaccid and an erect penis without need for any adhesive tape or other auxiliary clamping mechanisms to hold it in place. In contrast to those male external catheters that feature a similar sheath folding mechanism, the sheath not only keeps the device attached and seals against leakage, but takes actively part in the skin expansion process due to its material properties and its force distribution onto the shaft skin.

The presented skin-tube clamping and tensioning device furthermore overcomes stability-related problems of foldable male external catheters that do not withstand prolonged axial tensional forces without damage. This is achieved by mechanically anchoring a robust hollow pulling rod insert within the device instead of letting tensile forces act locally on fragile device elements (e.g., tubing) as it is the case for catheters. Contrary to the force distribution in catheters, the hollow pulling rod insert thus spreads the tensile load equally onto the entire device without increasing the compressive load onto the entrapped skin. The presented skin-tube clamping and tensioning device is therefore compatible with all published traction-generating strategies, be it manual stretching or based on weights or elastic straps, which can be attached to the hollow pulling rod insert, thereby allowing the user to select his preferred tension-generating method and to adjust the stretching force to his needs.

With the sturdy hollow pulling rod insert firmly engaged within the softer device body, the present skin-tube clamping and tensioning device forms a one-piece unit; there are no auxiliary parts that can fall off and get lost.

When compared with the sizes of any other non-surgical prior art foreskin clamping and stretching devices, the overall dimensions of the presented skin-tube clamping and tensioning device are distinctly smaller after application, thereby rendering it inconspicuous.

Furthermore, the presented skin-tube clamping and tensioning device allows for the leak-free passage and discharge of fluids from the urethral exit through the hollow pulling rod insert without requiring the user to take the device off. In addition, the device will not slip off when in contact with water during aquatic sport activities or when taking a bath or a shower.

Due to the skin-like softness, flexibility and biocompatibility of all materials in contact with the skin, there is furthermore a highly reduced risk of irritating or squeezing the skin too tightly, thus allowing for longer wearing periods without experiencing pain or causing tissue damage. For the same reason, the outside device surface will not scratch or otherwise irritate the skin of other body parts upon getting into contact with them when wearing the device. Furthermore, device translucency permits direct skin inspection to detect skin folds or other irregularities.

Other features and advantages of the presented skin-tube clamping and tensioning device will become apparent from the following, more detailed description, taken in conjunction with the accompanying drawings, which illustrate, by way of example, the principles of the invention.

### BRIEF DESCRIPTION OF DRAWINGS

The accompanying drawings illustrate the skin-tube clamping and tensioning device. In such drawings, arrows indicate objects, solid lines indicate regions, dashed lines indicate surface shapes.

FIG. 1 depicts the skin-tube clamping and tensioning device, without the hollow pulling rod insert present, in its folded and unfolded state, both in perspective, top and section views.

FIG. 2 depicts the skin-tube clamping and tensioning device, with the hollow pulling rod insert engaged, applied to a human penis without tension and under light tension in perspective, top and section views.

FIG. 3 depicts the hollow pulling rod insert in perspective, top and section views.

### DETAILED DESCRIPTION OF THE PREFERRED EMBODIMENT

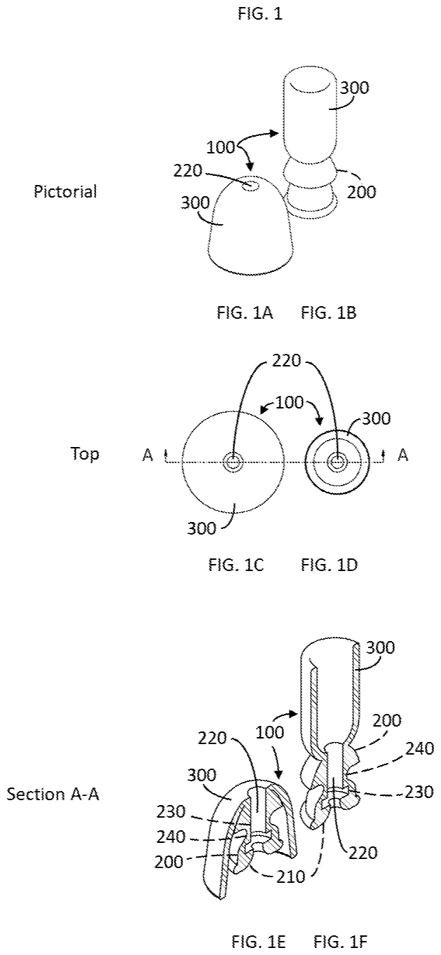

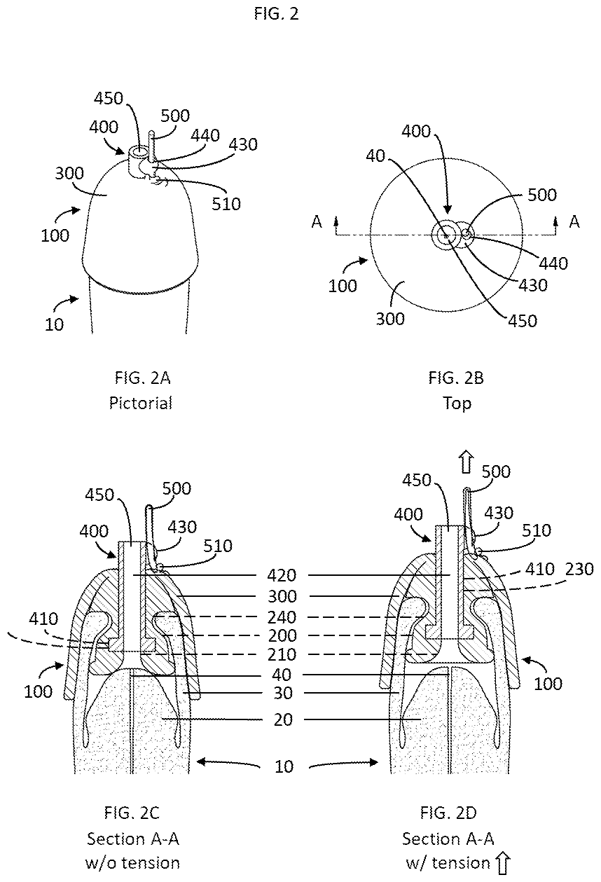

FIGS. 1 and 2 show a penile appliance made of a soft and stretchable material that serves as a skin-tube clamping and tensioning device for the human penile shaft skin or the foreskin.

A hollow body 100 provides a mushroom-shaped outer surface 200 at its base and a cylindrical protrusion 300 at its opposite end (FIG. 1B). The cylindrical protrusion 300 can be folded backward over the mushroom-shaped outer surface 200 of the hollow body 100 (FIG. 1A).

A penile skin tube 30 slides onto the stipe-portion of the mushroom-shaped outer surface 200 of the hollow body 100 in its unfolded state and is entrapped in a toroidal recess 240 upon turning the cylindrical protrusion 300 inside out, thereby folding it backwards towards the base of the hollow body 100 to rest with its originally outside surface on the outer surface of the skin tube 30 (FIG. 2A, C). As apparent to one skilled in the art, both the outer surface of the cylindrical protrusion 300 and the mushroom-shaped outer surface 200 of the hollow body 100 could furthermore be structured to enhance their grip to the entrapped skin tube 30. As furthermore apparent to one skilled in the art, the protrusion 300 can deviate from cylindrical shape towards either dome shape or funnel shape to better fit diverse penis geometries.

The axial length of the cylindrical protrusion 300 matches or exceeds the axial length of the mushroom-shaped outer surface 200. The largest outer diameter of the cylindrical protrusion 300 stays within the largest diameter of the user's glans 20. A material-dependent wall thickness is chosen for the cylindrical protrusion 300 that renders it stable and prevents its rupture when applying typical pulling forces during use.

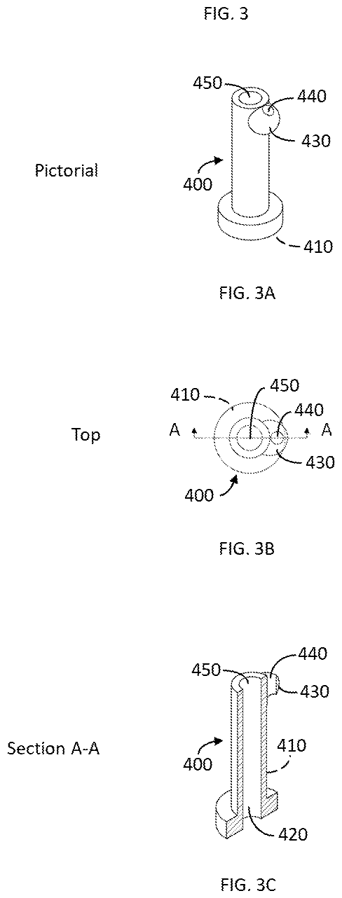

During application of the skin clamping and stretching device, a funnel-shaped inner surface 210 at the base of the hollow body 100 temporarily rests on the tip of the glans 20 of the penis 10 (FIG. 2C). An inside cavity 220 within the hollow body 100 at the end of the inner funnel 210 accommodates a rigid hollow insert 400 (FIG. 3, 2A-D). The surface of the inner funnel 210 of the hollow body 100 is smooth and slightly rounded at its entrance to form a liquid-tight seal with the glans 20 when both are in contact (FIG. 2C). Upon proper alignment with the urethral exit 40, the inner funnel 210 will guide urine into the device and direct it through the inner tubular passage 420 of the hollow insert 400 towards its exit 450, where it is discharged from the device.

The outer surface 410 of the hollow insert 400 and the inner surface 230 of the inner cavity 220 of the hollow body 100 both have matching shapes where in contact with each other to mechanically fasten the hollow insert 400 firmly within the hollow body 100 (FIG. 2C, D). Apparent to those skilled in the art, the matching surface shapes 230, 410 can be designed differently to optimize the mechanical anchoring of the hollow insert 400 within the hollow body 100 upon axial application of a pulling force.

The inner tubular passage 420 of the hollow insert 400 is smooth and has a diameter that matches the exit-diameter of the inner funnel 210 of the hollow body 100. This exit-diameter matches or exceeds the diameter of the urethral exit 40 (FIG. 2C, D).

A string 500 is slid through a through-hole 440 of an appendix 430 at the outside upper end of the hollow insert 400 to apply an axial pulling force onto the assembly of the hollow body 100 with the hollow insert 400 engaged therein (FIG. 2A-D). In the simplest case, a knot 510 at the lower end of the string 500 prevents the string from escaping the through-hole 440 upon applying an upward pulling force. As could be readily devised by one skilled in the art, any number of blocking entities can be attached to the string 500 to serve this purpose and to facilitate the exertion of a pulling force. Equally apparent to those skilled in the art, appendix 430 could have any other shape, relative position or fastening mechanism for strings or other kinds of pulling aids. Any pulling force that is axially applied to the string 500 will lift the assembly of the hollow body 100 and the hollow insert 400 from the glans 20, and axially stretch the skin 30 of the penis 10 (FIG. 2D).

The hollow body 100 is preferably made of a soft, highly elastic, translucent and biocompatible material. Apparent to those skilled in the art, an axial gradient in material stiffness is beneficial to physically reinforce the inner cavity 220 of the hollow body 100 while keeping the remaining device soft.

The hollow insert 400 is preferably made of a stiff, robust and biocompatible material such as a polymer, a metal or a combination of these two.

### CITATION LIST

### Patent Literature

GB2357976A, Richard Davey, Prepuce tissue expander, <URL:https://patents.google.com/patent/GB2357976A/en>
GB201305633D0, Gareth Paul Plant, Forenew foreskin restoration system, <URL:https://patents.google.com/patent/GB201305633D0/en>
GB2512403A, Gareth Paul Plant, Foreskin restoration device, <URL:https://patents.google.com/patentGB2512403A/en>
U.S. Pat. No. 6,579,227B2, James A. Haughey, Foreskin restoration device, <URL:https://patents.google.com/patent/U.S. Pat. No. 6,579,227B2/en>
U.S. Pat. No. 5,344,396A, Roland T. Clark, Jr., Device for stretching the foreskin of the penis, <URL:https://patents.google.com/patent/U.S. Pat. No. 5,344,396A/en>
USD375358S, Roland T. Clark, Penis foreskin stretching instrument, <URL:https://patents.google.com/patent/USD375358>
US20050090708A1, Ron Low, Tapeless device for retaining the foreskin of a circumcised penis over the glans, <URL:https://patents.google.com/patent/US20050090708>
WO2008019419A1, Rodney Rowell, A clamp to restore the foreskin, <URL:https://patents.google.com/patent/WO2008019419A1>
WO 2009/135268A1, Alan Watts & Andrew E. James, Foreskin stretching, <URL:https://patents.google.com/patent/WO2009135268A1>

### Patent Literature Cited by the International Searching Authority (ISA)

ISA-A U.S. Pat. No. 4,388,923 A, Ken Heimreid, External urinary drainage device, <URL: https://patents.google.com/patent/U.S. Pat. No. 4,388,923A>
ISA-B WO2001035877A1, Allan TANGHOJ, Henrik Lindenskov Nielsen, Signe Uhre Guldfeldt, An external urinary catheter assembly,
<URL:https://patents.google.com/patent/WO2001035877A1>

### Non-Patent Literature

Legend: [A] Journal article; [B] Book; [R] Report; [W] Website
[A1] Collier R., Whole again: the practice of foreskin restoration. CMAJ2011; 183(18):2092-2093. Doi:10.1503/cmaj.109-4009
[A2] Hvid I., Horn J., Huhnstock S., Steen H., The biology of bone lengthening. J Child Orthop. 2016 December; 10(6):487-492. Doi: 10.1007/s11832-016-0780-2
[A3] Wagh M S, Dixit V. Tissue expansion: Concepts, techniques and unfavourable results. Indian J Plast Surg. 2013 May; 46(2):333-48. Doi: 10.4103/0970-0358.118612
[B1] Jim Bigelow, PhD, “The Joy of Uncircumcising!” (2^{nd} ed.), Hourglass Book Publishing, ISBN 0-9630482-1-X
[R1] World Health Organization (WHO) and Joint United Nations Programme on HIV/AIDS (UNAIDS) (2007) Male circumcision: global trends and determinants of prevalence, safety and acceptability
[W1] Restoration Devices, The National Organization of Restoring Men (NORM); retrieved from the Internet <URL:http://www.norm.org/devices.html> on 2019-11-23
[W2] Restoration Product Comparison (updated 2019 Jan. 21); retrieved from the Internet <URL:https://www.x-mail.net/restore/compare.htm> on 2019-09-09; (alternate MS Excel chart comparison with links are made available by Schmeck W., (updated 2019 Aug. 22); retrieved from the Internet <URL:https://www.foreskin-restoration-devices.com/device-comparision> on 2019-09-09
[W3] Wikipedia—Tissue expansion; retrieved from the Internet <URL:https://en.wikipedia.org/wiki/Tissue_expansion> on 2019-08-28
[B2] V. Geng, H. Cobussen-Boekhorst, H. Lurvink, I. Pearce, S. Vahr, Male external catheters in adults—Urinary catheter management, European Association of Urology Nurses, 2016, ISBN: 978-90-79754-87-8

What is claimed is:

1. An appliance suitable for clamping and applying axial tension onto a human penile skin tube comprising a soft hollow body (100) with, at its proximal end, a funnel-shaped inner surface (210) and a mushroom-shaped outside surface (200) in contact with the inside surface of a penile skin tube (30) up to a toroidal recess (240) on its stipe, and with, at its distal end, a reversible hollow protrusion (300) in contact, in its backfolded state, with the outside surface of the penile skin tube (30), characterized in that said funnel-shaped inner surface (210) of said hollow body (100) extends into an inner cavity (220) that carries an axially hollow and rigid pulling rod insert (400) as discharge conduit, as structural stabilizer and as distributor of externally acting tensile loads.

2. The appliance according to claim 1, characterized in that the surface shape (230) of said inner cavity (220) matches the proximal outer surface shape (410) of said hollow pulling rod insert (400) over their entire contact length.

3. The appliance according to claim 1, characterized in that said inner cavity (220) interlocks said hollow pulling rod insert (400) by a tight mechanical fit.

4. The appliance according to claim 1, characterized in that said hollow pulling rod insert (400) has a predominantly cylindrical shape.

5. The appliance according to claim 1, characterized in that said hollow pulling rod insert (400) has at least one circumferential protrusion.

6. The appliance according to claim 1, characterized in that said hollow pulling rod insert (400) has an appendix (430) at its distal end adapted to attach an external pulling entity.
